(19) United States
(12) Patent Application Publication
(10) Pub. No.: US 2015/0297274 A1
(43) Pub. Date: Oct. 22, 2015

(54) LOCKING BUTTON FOR TAPE CERCLAGE

(71) Applicant: Arthrex, Inc., Naples, FL (US)

(72) Inventors: Peter J. Dreyfuss, Naples, FL (US); Thomas Dooney, JR., Naples, FL (US); Brian S. Cohen, Dublin, OH (US); Reuben Gobezie, Cleveland, OH (US)

(21) Appl. No.: 14/625,374

(22) Filed: Feb. 18, 2015

Related U.S. Application Data
(60) Provisional application No. 61/941,562, filed on Feb. 19, 2014.

(51) Int. Cl. A61B 17/82; A61B 17/132

(52) U.S. Cl. CPC A61B 17/82; A61B 17/1327

(57) ABSTRACT

A tensioning construct with a tape securely attached to a locking button provided with a securing mechanism in the form of a trap door-like mechanism. The tape is looped so that the looped portion of the tape is passed around a feature of the button to allow easy sliding of the tape. One free end of the tape is fed through the securing mechanism (trap door) of the locking button while the other free end is fixed to the locking button. The button self-engages without the need of a locking instrument, to lock the tape as it is tightened during the cerclage repair. The tensioning construct may be provided as a pre-assembled construct.

### CROSS-REFERENCE TO RELATED APPLICATIONS

This application claims the benefit of U.S. Provisional Application No. 61/941,562, filed Feb. 19, 2014, the entire disclosure of which is incorporated by reference herein.

### FIELD OF THE INVENTION

The present invention relates to the field of surgery and, more specifically, to apparatus and methods for reconstructive surgeries.

### BACKGROUND OF THE INVENTION

During treatment of fractures, it is often necessary to hold the bone or fragments of bone together, to create a stable environment for healing to occur. This is typically accomplished with metal wires or cables using a technique called cerclage. A cerclage wire or cable is wound around a bone or bony fragments to hold them together to allow them to heal.

Although wire cerclage has had numerous applications in orthopedics as a primary method of fracture fixation, it also has several disadvantages. Monofilament wires are prone to breakage while multi-filament cables are subject to fatigue and fraying, releasing metallic particulate debris into the body. The metal wire or cable can also break, particularly during tensioning, causing an interruption of the blood supply to the bone. The metal can also fray and fret causing tissue irritation and a source of blood-borne pathogen exposure to medical personnel. This is primarily why surgeons have begun to use simple sutures or ropes with metal fittings (for example, titanium fitting) in lieu of the wire cerclage.

There is a need for improved tensioning and securing of a tape cerclage during a tension band repair. Also needed are methods of adjusting the tension of a tape cerclage as the tape is being tightened during the repair.

### SUMMARY OF THE INVENTION

The present invention provides a tensioning construct with a flexible material securely attached to a locking button provided with a securing mechanism in the form of a trap door-like mechanism. The flexible material is looped so that the looped portion of the flexible material is passed around a feature of the button to allow easy sliding of the tape. One free end of the flexible material is fed through the securing mechanism (trap door) of the locking button while the other free end is fixed to the locking button. The button self-engages without the need of a locking instrument, to lock the tape as it is tightened during the cerclage repair. The tensioning construct may be provided as a pre-assembled construct.

The present invention also provides a method of providing a compressive force across a repair (bone fragments or body tissues, etc.) by inter alia the steps of: (i) providing a pre-assembled construct comprising a flexible material securely attached to a locking button provided with a securing mechanism (a trap door); (ii) passing the loop end of the flexible material around the tissue (bone or soft tissue) and assembling the loop onto the button feature; and (iii) pulling on the free end of the flexible material to tension the entire construct and fix it at the same time.

These and other features and advantages of the invention will be more apparent from the following detailed description that is provided in connection with the accompanying drawings and illustrated exemplary embodiments of the invention.

### BRIEF DESCRIPTION OF THE DRAWINGS

FIG. 1 illustrates a pre-assembled locking button construct with tape of the present invention.

FIG. 2 is a perspective view of the locking button of the construct of FIG. 1.

FIG. 3 is a perspective view of a locking button according to another embodiment of the present invention.

### DETAILED DESCRIPTION OF THE PREFERRED EMBODIMENTS

In the following detailed description, reference is made to various specific embodiments in which the invention may be practiced. These embodiments are described with sufficient detail to enable those skilled in the art to practice the invention, and it is to be understood that other embodiments may be employed, and that structural changes may be made without departing from the scope of the present invention.

The present invention provides a tensioning construct and methods of cerclage repair employing such tensioning construct, which allow improved tensioning and securing of the construct during a tension band repair, without the risk of wire breaking and associated damage to body tissue and/or medical personnel.

The present invention provides a tensioning construct with a flexible material (for example, suture, suture tape and/or suture chain) securely attached to a locking button provided with a securing mechanism in the form of a trap door-like mechanism. The flexible material is looped so that the looped portion of the flexible material is passed around a feature of the button to allow easy sliding of the tape. One free end of the flexible material is fed through the securing mechanism (trap door) of the locking button while the other free end is fixed to the locking button. The button self-engages without the need of a locking instrument, to lock the tape as it is tightened during the cerclage repair. The tensioning construct may be provided as a pre-assembled construct.

The present invention also provides a cerclage device, comprising: (i) a locking button having a body with a general rectangular configuration, a longitudinal axis, a length, a width, and a height, a first end and a second end opposite the first end, the locking button further comprising a securing mechanism that allows a flexible material to be secured thereto, and an opening or slot that allows an end of the flexible material to be secured thereto; and (ii) a flexible material (for example, suture or suture tape) securely attached to the locking button and forming a loop. The locking button further comprises a plurality of slots or openings (for example, two slots or openings) located symmetrically relative to the longitudinal axis of the body, to allow the loop to slide therethrough.

As detailed below, the securing mechanism is preferably a trapdoor with a leaf spring that allows sliding and locking of the suture tape through the trapdoor. The suture tape has a width about equal to or smaller than a width of the trapdoor. In an exemplary embodiment, the securing mechanism and the opening or slot are located at one of the first end or second end. The locking button may be formed of metal or similar biocompatible materials. The securing mechanism is preferably formed integral with the body of the locking button. In an exemplary embodiment, the flexible material (tape) is pre-assembled on the locking button.

The present invention also provides a method of providing a compressive force across a repair (bone fragments or body tissues, etc.) by inter alia the steps of: (i) providing a pre-assembled construct comprising a flexible material (tape) securely attached to a locking button provided with a securing mechanism (a trap door); (ii) passing the loop end of the flexible material around the tissue (bone or soft tissue) and assembling the loop onto the button feature; and (iii) pulling on the free end of the flexible material to tension the entire construct and fix it at the same time.

According to another exemplary embodiment, the present invention provides a method of tension band repair by inter alia the steps of: (i) providing a first tissue segment adjacent a second tissue segment; (ii) providing a pre-assembled cerclage construct comprising a locking button and a loop of a suture tape attached to the locking button, the locking button having a body with a length, a width and a height, a longitudinal axis, a first end and a second end opposite the first end, the locking button further comprising a securing mechanism that allows the suture tape to be secured thereto, and an opening or slot that allows an end of the suture tape to be secured thereto, wherein the pre-assembled cerclage construct is provided by securing a first end of the suture tape to the opening or slot and passing a second end of the suture tape through the securing mechanism to form the loop; and (iii) passing the loop of the suture tape around the first and second tissue segments to reduce a distance between the first and second tissue segments.

As detailed below, the method of tension band repair may further comprise the steps of (iv) assembling the loop of the suture tape onto the locking button by passing the loop through two openings provided within the body of the locking button, the openings extending from an outer surface of the body of the locking button and within the body of the locking button; (v) pulling the second end of the suture tape after the step of passing the second end of the suture tape through the securing mechanism; and (vi) tensioning and fixing the cerclage construct, wherein the steps of tensioning and fixing the cerclage construct are conducted simultaneously.

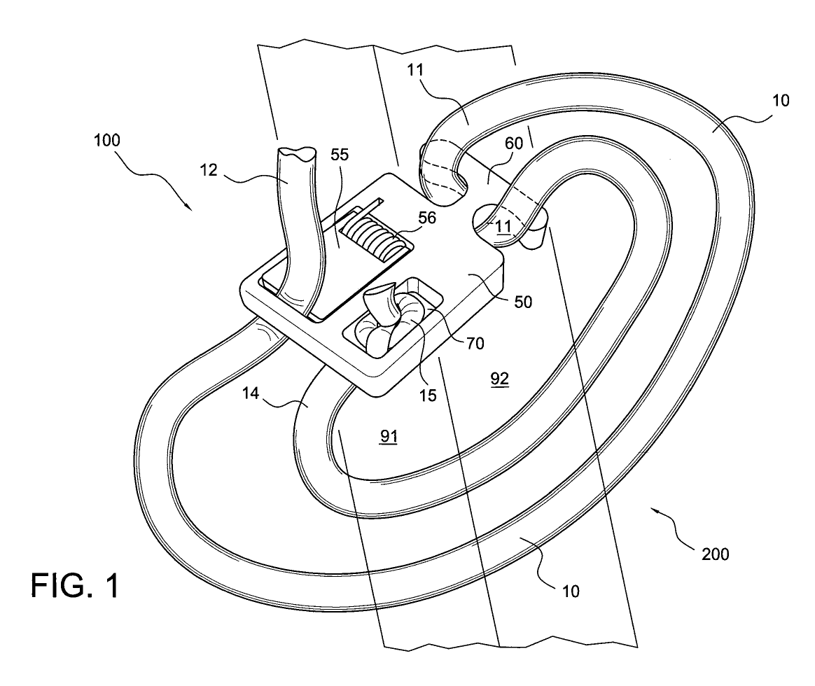

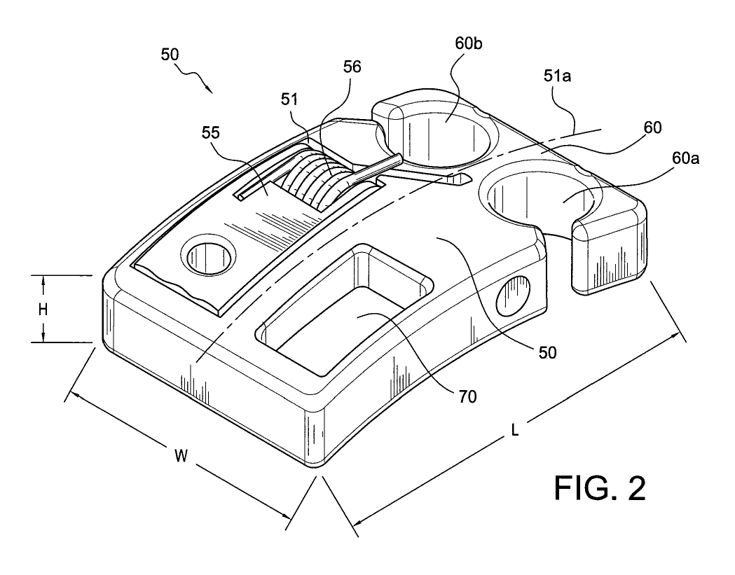

FIG. 1 illustrates exemplary construct 100 (cerclage device or cerclage construct 100) of the present invention including exemplary locking button 50 and flexible material 10 assembled to the button and provided schematically around two tissue segments 91, 92 (for example, two bone fragments 91, 92) to achieve repair 200. FIG. 2 illustrates details of the locking button 50. As detailed below, the flexible material 10 may be suture, suture tape, suture chain or combinations of these exemplary materials. Preferably, the flexible material 10 is tape such as suture tape.

Locking button 50 is preferably formed of metal or similar material. Locking button 50 is provided with a body 51 having a general overall rectangular configuration, with a length L, a width W and a height H, and a longitudinal axis 51a (shown more clearly in FIG. 2).

Locking button is also provided with a securing mechanism 55 in the form of a trap door 55, a feature 60 that allows flexible material 10 (tape 10) to be looped around, and an opening or slot 70 that allows an end of the flexible material 10 (tape 10) to be securely affixed thereto, and as described below.

The flexible material 10 (tape 10) is looped to form a looped portion 11 and two free ends 12, 14. The looped portion 11 of the tape 10 is passed around feature 60 of the button 50 to allow easy sliding of the tape. Free end 12 of the tape is fed through the securing mechanism 55 (trap door 55) with a leaf spring 56 of the locking button, while the other free end 14 is fixed to the opening/slot 70 of the locking button 50, by forming a knot 15, for example, or by other known methods of attachment.

In an exemplary-only embodiment, the feature 60 is in the form of two openings or slots 60a, 60b extending through the body 51 of the locking button, and from one surface to an opposite surface, for example, from a top surface to a bottom surface of the body 51, and also communicating with a side surface (perimeter) of the body 51. The two openings or slots 60a, 60b extend from a side surface of the body 51 and into the body. The two openings or slots 60a, 60b are located about symmetrically relative to the longitudinal axis 51a of the body 51.

The button 50 is self-engaging, without the need of a locking instrument, to lock the flexible material 10 (tape 10) as it is tightened during a cerclage repair. The tensioning construct 100 may be provided as a pre-assembled construct. The securing mechanism 55 (trap door 55 with a leaf spring 56) is provided as a unitary construct/feature with the locking button.

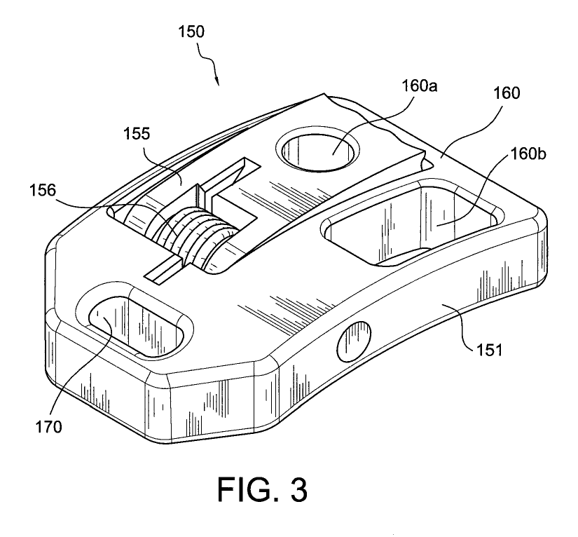

FIG. 3 illustrates another exemplary embodiment of locking button 150 of the present invention, which is about similar to locking button of FIGS. 1 and 2 but differs in design characteristics. Locking button 150 is also preferably formed of metal or similar material and is provided with a body 151 that is integral with a securing mechanism 155 in the form of a trap door 155, a feature 160 that allows flexible material 10 (tape 10) to be looped through it and attached to the button, and an opening or slot 170 that allows an end of the flexible material 10 (tape 10) to be securely affixed thereto, and as described above with reference to slot 70.

As in the previously-described embodiment, tape 10 is passed through feature 160 of the button 150 and then free end 12 of the tape is fed through the securing mechanism 155 (trap door 155) with a leaf spring 156 of the locking button, while the other free end 14 is fixed to the opening/slot 170 of the locking button 150, by forming a knot, for example, (not shown) or by other known methods of attachment.

In an exemplary-only embodiment, the feature 160 is in the form of two through openings or through slots 160a, 160b extending through the body 151 of the locking button, and from one surface to an opposite surface, for example, from a top surface to a bottom surface of the body 151. The two openings or slots 160a, 160b have different geometries (as shown in FIG. 3) but could also have similar shape/geometry.

The button 150 is self-engaging, without the need of a locking instrument, to lock the flexible material 10 (tape 10) as it is tightened during a cerclage repair. As in the previous embodiment, the button 150 may be provided as a pre-assembled construct, with tape or flexible material attached to it. The securing mechanism 155 (trap door 155 with a leaf spring 156) is provided as a unitary construct/feature with the locking button.

The present invention also provides a method of providing a compressive force across a repair (bone fragments or body tissues, etc.) by inter alia the steps of: (i) providing a pre-assembled construct 100 comprising a suture tape 10 securely attached to a locking button 50, 150 provided with a securing mechanism 55, 155 (a trap door 55, 155) that allows sliding and locking of the suture tape 10; (ii) passing the loop end 11 of the tape 10 around the tissue (bone, bone fragments or soft tissue) and assembling the loop onto button feature 60, 160; and (iii) pulling on the free end 12 of the tape 10 to tension the entire construct 100 and fix it at the same time.

Flexible material 10 may be in the form of any suture, tape or chain, or combinations thereof. In an exemplary embodiment only, flexible material 10 is in the form of a tape which may include a wide selection of synthetic compositions such as ultrahigh molecular weight polyethylene (UHMWPE), or the FiberWire® suture described in U.S. Pat. No. 6,716,234, or the FiberTape® suture tape described in U.S. Pat. No. 7,892,256, the disclosures of both of which are incorporated by reference in their entirety herewith. FiberTape® is a suture tape (flat braid) with an ultra-high strength, about 2 mm wide, and with a structure similar to that of the FiberWire® suture. The tape provides broad compression and increased tissue cut-through resistance making it a preferred choice for knotless rotator cuff repairs or for high demand applications like AC joint reconstruction and other areas where tensioning with conventional wires may be a concern. Preferably, the tape is FiberTape® of about 2 mm wide, which is about equal to the width of the trapdoor 55, 155.

The tape 10 may be also provided as a flat braid formed of braided fibers, for example, braided ultrahigh molecular weight polyethylene fiber blended with fibers of one or more long chain synthetic polymers, preferably polyester.

The construct 100 of the present invention may be employed in many surgical applications that require conventional cerclage cabling for fixation and treatment, for example, in femoral fractures (i.e., fractures around a hip stem), hip fractures, humerus/shoulder fractures, patella fractures, elbow fractures, etc. Construct 100 may be also employed in various other fracture repairs and arthroplasties, such as sternotomies, AC joint repairs, and many other indications that require tensioning of the repair, and may have application to the repair of any number of tissue pieces (i.e., two or more tissue fragments/pieces). Repair 200 shown schematically in FIG. 1 is only an illustrative embodiment showing two tissue pieces 91, 92 that are brought together (i.e., reducing the distance between adjacent tissue pieces).

Although the present invention has been described in connection with preferred embodiments, many modifications and variations will become apparent to those skilled in the art. While preferred embodiments of the invention have been described and illustrated above, it should be understood that these are exemplary of the invention and are not to be considered as limiting. Accordingly, it is not intended that the present invention be limited to the illustrated embodiments, but only by the appended claims.

What is claimed as new and desired to be protected by Letters Patent of the United States is:

1. A cerclage device, comprising:
a locking button having a body with a general rectangular configuration, a longitudinal axis, a first end and a second end opposite the first end, the locking button further comprising a securing mechanism that allows a flexible material to be secured thereto, and an opening or slot that allows an end of the flexible material to be secured thereto; and
a flexible material securely attached to the locking button.

2. The cerclage device of claim 1, wherein the locking button further comprises a plurality of slots or openings located symmetrically relative to the longitudinal axis of the body.

3. The cerclage device of claim 1, wherein the slots or openings allow the flexible material to slide around the slots or openings.

4. The cerclage device of claim 1, wherein the securing mechanism is a trapdoor with a leaf spring that allows sliding and locking of the flexible material through the trapdoor.

5. The cerclage device of claim 4, wherein the flexible material is suture tape having a width about equal to a width of the trapdoor.

6. The cerclage device of claim 1, wherein both the securing mechanism and the opening or slot are located at one of the first and second ends.

7. The cerclage device of claim 1, wherein the locking button is formed of metal.

8. The cerclage device of claim 1, wherein the securing mechanism is formed integral with the body of the locking button.

9. The cerclage device of claim 1, wherein the flexible material is suture, suture tape or suture chain.

10. The cerclage device of claim 1, wherein the flexible material is formed of suture material comprising ultrahigh molecular weight polyethylene.

11. The cerclage device of claim 1, wherein the flexible material is pre-assembled on the locking button.

12. A method of tension band repair, comprising the steps of:
providing a first tissue segment adjacent a second tissue segment;
providing a pre-assembled cerclage construct comprising a locking button and a loop of a suture tape attached to the locking button, the locking button having a body with a length, a width and a height, a longitudinal axis, a first end and a second end opposite the first end, the locking button further comprising a securing mechanism that allows the suture tape to be secured thereto, and an opening or slot that allows an end of the suture tape to be secured thereto, wherein the pre-assembled cerclage construct is provided by securing a first end of the suture tape to the opening or slot and passing a second end of the suture tape through the securing mechanism to form the loop; and
passing the loop of the suture tape around the first and second tissue segments to reduce a distance between the first and second tissue segments.

13. The method of claim 12 further comprising the step of assembling the loop of the suture tape onto the locking button by passing the loop through two openings provided within the body of the locking button, the openings extending from an outer surface of the body of the locking button and within the body of the locking button.

14. The method of claim 12 further comprising the steps of pulling the second end of the suture tape after the step of passing the second end of the suture tape through the securing mechanism; and tensioning and fixing the cerclage construct.

15. The method of claim 14, wherein the steps of tensioning and fixing the cerclage construct are conducted simultaneously.

16. The method of claim 12, wherein the securing mechanism is a trapdoor with a leaf spring.

17. The method of claim 12, wherein the cerclage construct is a unitary construct.
